(19) United States
(12) Patent Application Publication
(10) Pub. No.: US 2022/0323073 A1
(43) Pub. Date: Oct. 13, 2022

(54) DEVICES AND METHODS FOR REDUCING THE APPEARANCE OF CELLULITE

(71) Applicant: TAUTONA GROUP IP HOLDING COMPANY, L.L.C., Redwood City, CA (US)

(72) Inventors: Geoffrey C. GURTNER, Woodside, CA (US); Michael H. ROSENTHAL, Menlo Park, CA (US); Joseph R. RIMSA, Palo Alto, CA (US)

(21) Appl. No.: 17/640,359

(22) PCT Filed: Sep. 4, 2020
(86) PCT No.: PCT/US2020/049421

§ 371 Date: Mar. 4, 2022

Related U.S. Application Data
(60) Provisional application No. 62/896,479, filed on Sep. 5, 2019.

(51) Int. Cl. A61B 17/08; A61B 17/3209; A61B 17/10

(52) U.S. Cl. CPC A61B 17/085; A61B 17/32093; A61B 17/10; A61B 2017/00747

(57) ABSTRACT

Disclosed herein are methods and devices for the treatment of cellulite. The methods comprise cutting the septae connecting the dermis from the underlying fascia, reducing the appearance of dimples and applying a patch to the skin over the cut septae to hold in place or remodel the skin to allow it to heal in the desired shape and to minimize the recurrence of dimples.

### CROSS REFERENCE TO RELATED APPLICATIONS

This application claims the benefit of U.S. Provisional Patent Application No. 62/896,479, filed Sep. 5, 2019, and entitled “DEVICES AND METHODS FOR REDUCING THE APPEARANCE OF CELLULITE”, the entire disclosure of which is incorporated by reference herein.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

This application relates to the field of treatment of cellulite.

### BACKGROUND

Cellulite is the herniation of subcutaneous fat within fibrous connective tissue that manifests topographically as skin dimpling and nodularity, often on the pelvic region (specifically the buttocks), lower limbs, and abdomen. Ninety five percent of women report having cellulite. Nearly fifty percent of 200,000 liposuction procedures performed in 2010 presented with cellulite. The US spends nearly $6 billion in cosmeceutical products and garments to ‘treat’ cellulite. In spite of this large demand, no available technology has shown consistently positive results. Currently available products are estimated to be less than fifty percent effective. The most promising clinical treatments are based on the healing of subcision intervention.

Subcision is a surgical technique used for the treatment of cutaneous depressions. It involves the use of a sharp instrument such as a needle, scalpel or “pickle fork”. A small incision or puncture is used to gain access to the subcutaneous space. The instrument is then used to delaminate the dermis from the muscle fascia by severing the septae that tether the shallow dermal layer to the underlying muscle structures, shown in FIG. 1.

Studies involving cadaveric dissection and non-invasive imaging have shown that cellulite lesions are most often depressions in the cutaneous surface related to thin vertical septa connecting the deep dermis to the fascia coexisting with large fat deposits extending into the reticular dermis. Subcision was specifically reported for the treatment of cellulite and liposuction sequella by Hexsel and Mazzuco in a series of case studies including 232 patients that sowed a moderate improvement in large dimples in the cellulite area. In addition, a study demonstrated that significantly thicker subcutaneous fibrous septa are present in areas with cellulite compared to areas without cellulite. These studies and findings indicated a need for a device specifically designed for selective subcision and severing of potentially differentiated septae.

While such treatment can reduce the appearance of cellulite, at least temporarily, it has been found that the cellulite will reform in the treated area. Thus, there remains a need to improve upon subcision treatment of cellulite.

### SUMMARY OF THE DISCLOSURE

In a first aspect, a patch for treatment of cellulite is provided. The patch comprises a flexible pad comprising a central portion and a border portion, the border portion thinner than the central portion; a backbone embedded within the flexible pad, the backbone more rigid than the flexible pad; and an adhesive configured to adhere the patch to skin, wherein the patch is configured to be adhered to the skin, thereby holding the skin in a remodeled configuration during healing.

In some embodiments, the backbone comprises a sheet of material comprising a plurality of apertures. The backbone can comprise a honeycomb structure. In some embodiments, the flexible pad comprises a silicone elastomer. The backbone can comprise a polycarbonate. In some embodiments, the adhesive comprises a pressure sensitive adhesive. The patch can be shaped to be positioned adjacent to other same shaped patches to form a matrix. In some embodiments, the patch comprises a backing positioned between the pad and the adhesive. The backing can comprise PTFE. In some embodiments, the patch comprises a plurality of apertures extending through the patch. The thickness of the flexible pad can be about 0.11-0.15 in. In some embodiments, a thickness of the border is about 0.026-0.026 in.

In another aspect, a method of reducing an appearance of cellulite is provided. The method comprises adhering a patch to skin over a treatment area of a user, the treatment area comprising released septae; and maintaining a configuration of the skin with the patch.

In some embodiments, the released septae are subcisioned or released through cutting or through electromechanical means. The patch can comprise a flexible pad comprising a central portion and a border portion, the border portion thinner than the central portion and a backbone embedded within the flexible pad, the backbone more rigid than the flexible pad.

In yet another aspect, an applicator for applying a patch to skin is provided. The applicator comprises a handle; and a body attached to the handle, the body comprising a top surface and a bottom applicator surface, the body comprising a plurality of apertures extending from the top surface to the bottom applicator surface, the bottom applicator surface curving towards the handle.

In some embodiments, the applicator comprises one or more indentations on the applicator surface shaped to receive the patch. The applicator can comprise a feature on the top surface configured to receive a vacuum source. In some embodiments, the body comprises an optically clear material. The applicator can be configured to apply embodiments of patches/splints described herein.

In still another aspect, a method of applying a patch to skin is provided. The method comprises providing an applicator comprising a body and a handle extending across the body; attaching a patch to a curved bottom applicator surface of the body; gripping the handle; applying downward force to the applicator; and rolling the applicator across the skin.

In some embodiments, attaching the patch to the curved bottom applicator surface of the body comprises applying vacuum to the patch. Attaching the patch to the curved bottom applicator surface of the body can comprise placing the patch within indentations of the bottom applicator surface. In some embodiments, the indentations are shaped to match a shape of the patch.

In another aspect, a targeting device for determining a cellulite treatment location is provided. The device comprises a handle; and a viewing portion attached to the handle, the viewing portion comprising a sheet of optically clear material.

In some embodiments, the handle comprises indentations shaped to receive a user's fingers. The viewing portion can have holes through which the therapist can use a marking pen to mark the treatment area. The sheet can be flat or curved.

In still another aspect, a method of determining a skin treatment location is provided. The method comprises holding a handle of a targeting device; and pressing a viewing portion of the targeting device against a potential treatment site, the viewing portion comprising a sheet of optically clear material.

In some embodiments, the method comprises selecting the potential treatment site and treating a skin defect at the treatment site.

In another aspect, a method of reducing an appearance of cellulite is provided. The method comprises selecting a treatment site in the user; cutting targeted septae at the treatment site; and adhering a patch to the skin over the treatment site, the patch comprising a flexible pad comprising a central portion and a border portion, the border portion thinner than the central portion and a backbone embedded within the flexible pad, the backbone more rigid than the flexible pad.

In some embodiments, selecting a treatment site in the user comprises holding a handle of a targeting device; and pressing a viewing portion of the targeting device against a potential treatment site, the viewing portion comprising a sheet of optically clear material. In some embodiments, adhering the patch to the skin over the treatment site comprises providing an applicator comprising a body and a handle extending across the body; attaching the patch to a curved bottom applicator surface of the body; gripping the handle; applying downward force to the applicator; and rolling the applicator across the skin. Attaching the patch to the curved bottom applicator surface can comprise applying vacuum to the patch. In some embodiments, an external vacuum can be used to pull the tissue into the patch through holes in the patch. The method can comprise adhering a second patch adjacent to the patch, the second patch placed such that edges of the patch and second patch align with one another.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1 illustrates anatomy of cellulite.

FIGS. 2A-2D illustrate various embodiments of targeting devices.

FIG. 3 shows a device for cutting tissue that can also perform liposuction

FIG. 4 is a cross-sectional view of the device of FIG. 1 with the cutting element collapsed

FIG. 5 is a cross-sectional view of the device of FIG. 1 with the cutting element expanded

FIG. 6 is a perspective view of the cutting element

FIG. 7 shows a number of tunnels created in tissue

FIG. 8 shows the cutting element deployed

FIG. 9 shows the cutting element moved to cut tissue

FIG. 10 shows the device advanced into the area where the tissue has been cut

FIG. 11 shows another device for cutting tissue

FIG. 12 shows a cross-sectional view of the device of FIG. 11 with the cutting element expanded

FIG. 13 shows a cross-sectional view of the device of FIG. 9 with the cutting element collapsed

FIG. 14 shows another device for cutting tissue.

FIG. 15 is a cross-sectional view of a deflecting element and the cutting element of the device of FIG. 14.

FIG. 16 shows the element of FIG. 15 deflected by a septae.

FIG. 17 shows another element which may be used with the device of FIG. 9.

FIGS. 18A-18D show another embodiment of a cutting device.

FIGS. 19A-19C show various views of embodiments of a patch.

FIG. 20 shows an embodiment of a matrix of patches.

FIGS. 21A-21C show embodiments of an applicator for applying patches.

FIGS. 22A-22D show temperature plots of a patch in compression and extension.

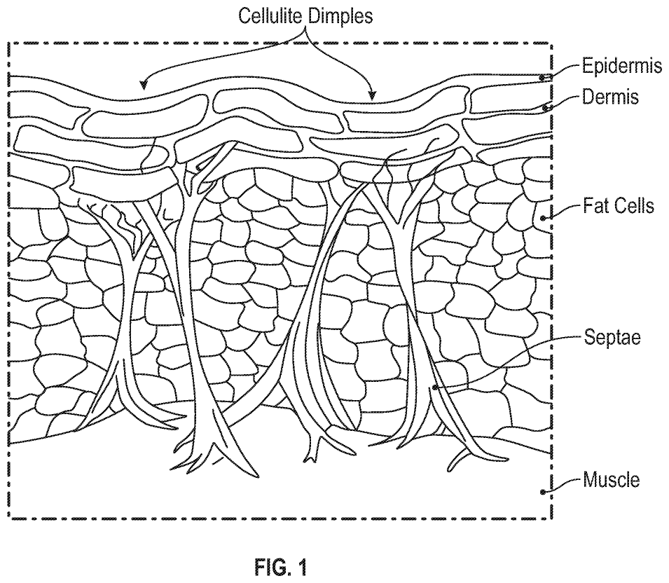

### DETAILED DESCRIPTION

Disclosed herein are devices and methods for reducing the appearance of cellulite. The method can comprise targeting or locating a treatment site or specific septae to be cut, cutting the septae, and applying a splint or patch to hold the skin in a desired configuration during healing.

It will be appreciated that these devices and methods may be applicable for treatment of other conditions involving irregular surface qualities of the skin (e.g. scarring, pitting, and wrinkles).

The general approach can include the following physiologic assumptions regarding the causes of the irregular skin surface topography most prominent in cellulite: (1) Fibrous septae connect the dermis to the underlying fascia, and these fibrous septae apply a force orthogonal to the skin plane creating dimples (2) With time the skin seems to become looser due to the effect of stresses on elastic fibers and collagen fibers in the skin. Gravity plays a large part in pulling the looser skin downwards (3) Fat, or adipocyte, accumulations in pockets surrounding the septae overcome the skins ability to prevent local pouching of the skin surface.

### Targeting a Treatment Location

A clinician performing the procedure described herein can first determine what area to treat. In some embodiments, the clinician may perform a visual inspection to focus on a treatment area. For example, the clinician may observe the largest or deepest dimples and choose to treat in that area.

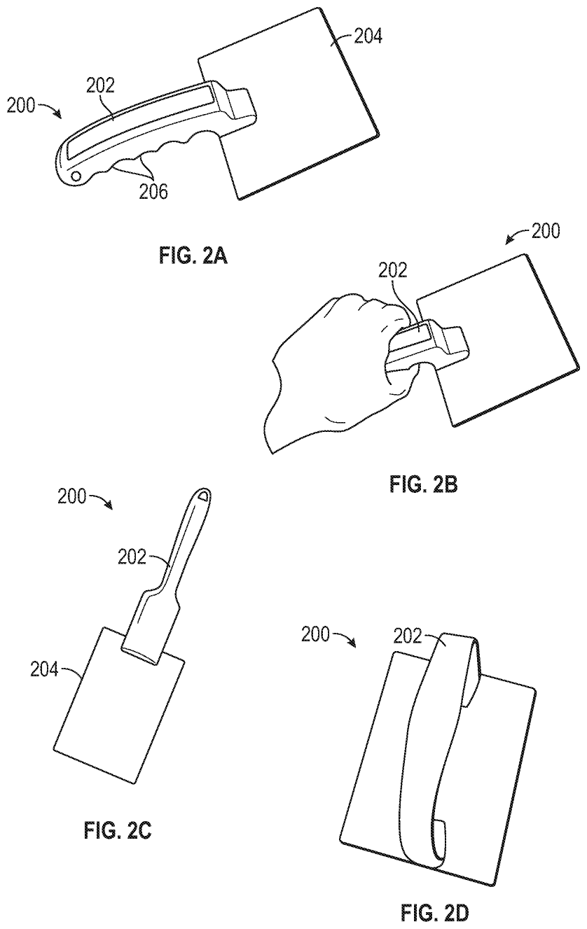

In some embodiments, a clinician may use a tool to identify and/or target a location to be treated. FIGS. 2A-2C show embodiments of a targeting device 200. As shown in FIG. 2A, the targeting device can comprise a handle 202 and a viewing portion 204. The viewing portion comprises a flat sheet of optically clear material. In some embodiments, the sheet of material can be curved. A clinician can use the targeting device 200 to press down on an affected area, flattening the skin beneath the viewing portion 204 to highlight the most significant dimples and determine a treatment location. The dimples would appear as “air pockets” below the rigid transparent paddle when downward force is applied to the skin. Other configurations of a viewing portion are also possible (e.g., ovular, circular, etc.) The handle 202 is attached to the viewing portion 204. In some embodiments, the handle is configured for gripping in one hand, as shown in FIG. 2B. The handle 202 is slightly curved and has indentations 206 shaped to receive a user's fingers while gripping the handle 202. FIG. 2C shows another embodiment of a targeting device 200. This device 200 also comprises a handle 202 and a viewing portion 204. The viewing portion is slightly longer and narrower than that shown in FIG. 2B. The different dimensions of the viewing portions can be used for different treatment locations. For example, a larger viewing portion can be used on a larger posterior section of the buttocks, while a smaller viewing portion may be more appropriately sized for a side section of the hips. FIG. 2D shows another embodiment of a targeting device 200 comprising a handle 202 and a viewing portion 204. The handle 202 is connected at either end of the viewing portion 204. This configuration may be more suitable for applying more force, for example, in an area with deep cellulite. The handles 202 of FIGS. 2A and 2C may be more appropriate for shallower cellulite requiring more careful inspection as the handle does not cover the viewing area. It will be appreciated that other configurations of a targeting device are also possible, as long as they include a viewing portion that can be pressed against affected skin.

In some embodiments, the targeting device 200 can also be used after cutting the septae to assess the efficacy of the treatment and determine which other areas, if any, should be treated.

### Cutter

Embodiments of cutters are provided in U.S. Pat. No. 8,652,123, filed on Sep. 2, 2008, the entire disclosure of which is hereby incorporated by reference in its entirety.

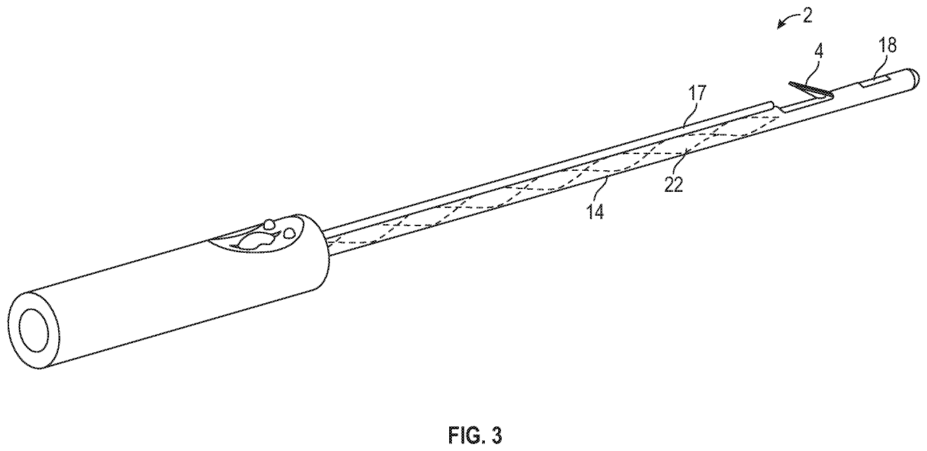

Referring to FIGS. 3-5, a tissue cutting device 2 is shown. The device 2 is particularly suited for cutting septae while performing a liposuction procedure as described below.

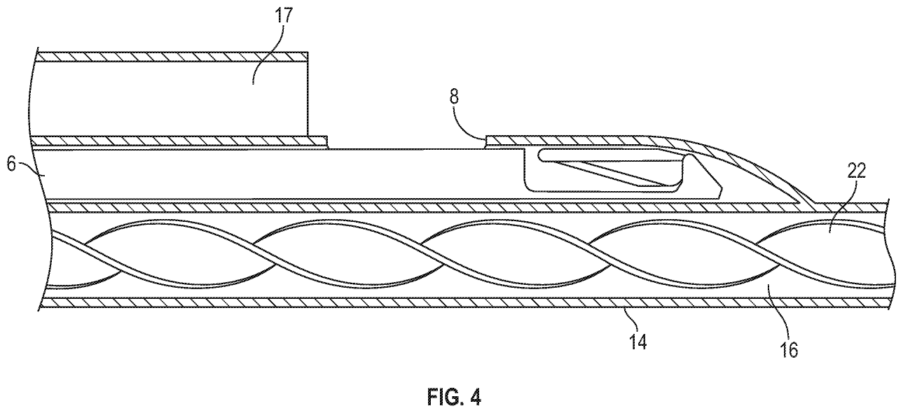

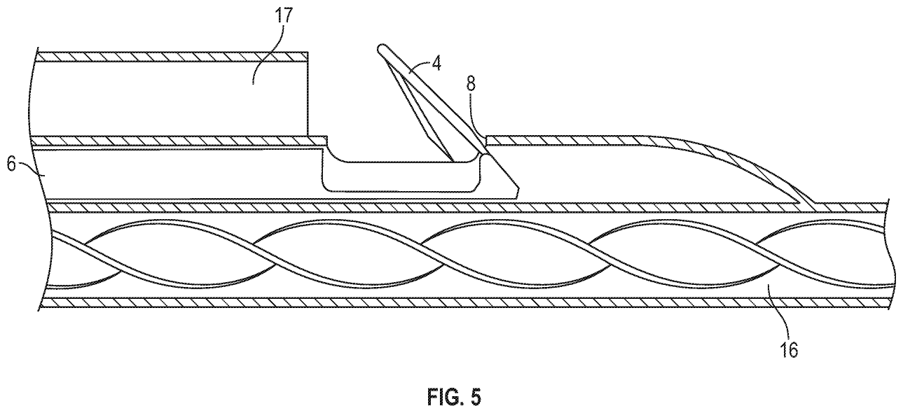

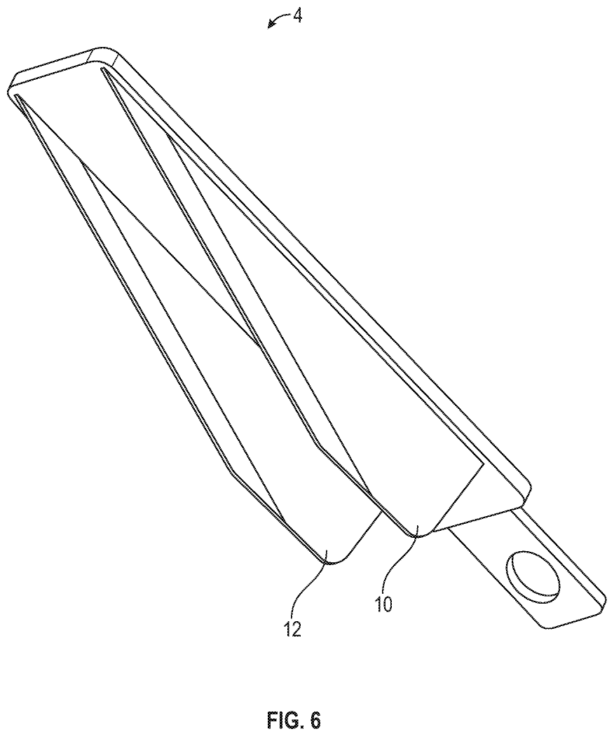

The device 2 includes a mechanical cutting element 4 to cut tissue although any other suitable cutting element 4 may be used. The cutting element 4 is movable from the collapsed position of FIG. 4 to the expanded or cutting position of FIG. 5. The cutting element 4 is coupled to a push rod 6 which is manipulated to move the cutting element 4 between the stored and cutting positions. The cutting element 4 is naturally biased toward the expanded position and is collapsed by advancing the push rod so that the cutting element 4 engages a shoulder 8 to force the cutting element 4 back to the collapsed position as the rod 6 is advanced. Referring to FIG. 6, a perspective view of the cutting element 4 is shown. The cutting element 4 may have a first blade 10 and a second blade 12 to remove a section of the septae. Removal of a section, rather than simply cutting the septae at one location, may help to prevent the septae from reattaching during the healing process. The first and second blades 10, 12 may be separated by at least 0.5 mm. It is understood that numerous aspects of the present invention may be practiced with different cutting elements such as RF, ultrasound or laser, or a single cutting element rather than two blades without departing from the scope of the invention. Furthermore, cutting may be accomplished using blunt dissection as described further below.

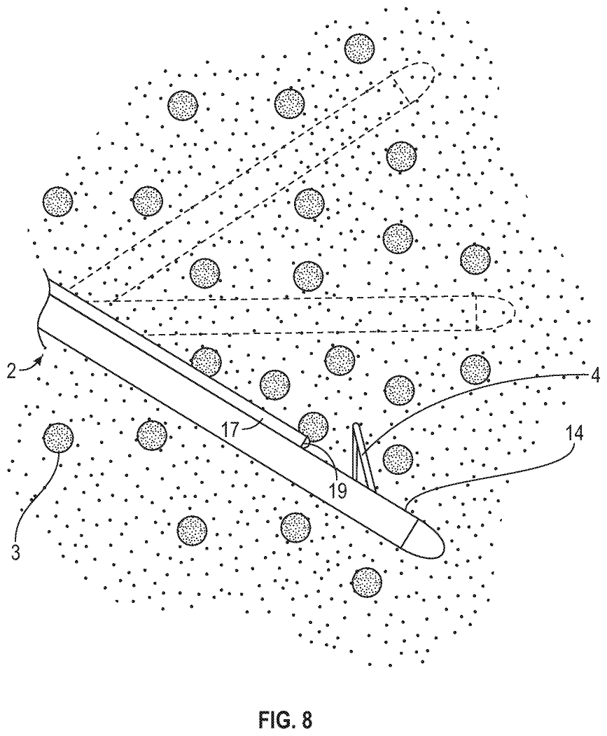

The device 2 includes an elongate body 14 having a suction lumen 16 extending there through. The suction lumen 16 leads to one or more suction openings 18 along the length of the body 14. The suction lumen 16 is coupled to a source of suction so that suction may be used to draw fat and other tissue into the suction openings as is known in conventional liposuction. A rotating element 22, which may be shaped similar to an auger, may be positioned in the suction lumen 16 to assist removal of tissue. The device 2 may also have a lumen 17 which receives a visualization device 19 (see FIG. 8) such as an endoscope 21. The lumen 17 is positioned so that the user may view the tissue captured by the cutting element 4. The visualization device 19 may also be used to inspect the tissue prior to cutting. In this manner, the user may deploy the cutting element 4 to cut septae and retract the element 4 when encountering large blood vessels or nerves. The endoscope can further allow visualization of the distal tip of the cannula, which can prevent severe complications associated with undesirably puncturing body cavities.

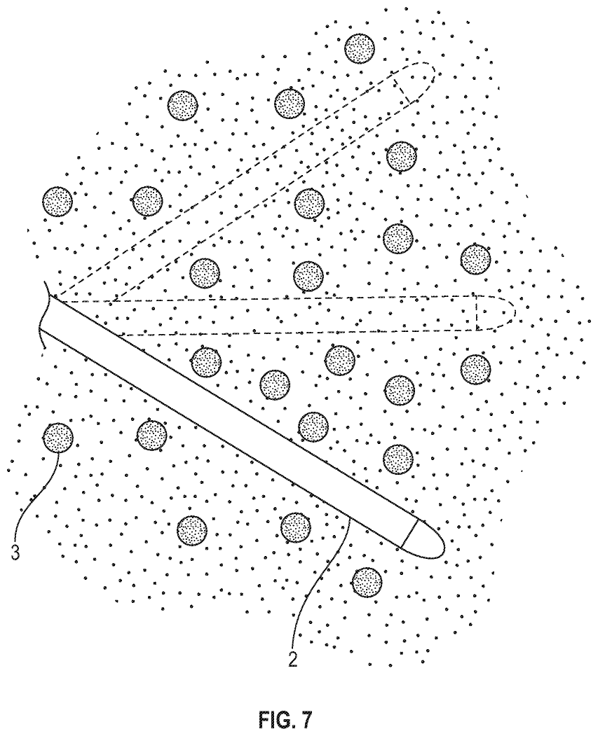

As mentioned above, a problem with conventional liposuction is that the liposuction wand will naturally be limited to a number of passageways or tunnels as shown in FIG. 7. It is often difficult to access the areas between these tunnels since the liposuction cannula will take the path of least resistance rather than penetrating new tissue. In particular, the septae 3 which interconnect the skin and muscle can be difficult to penetrate and can limit mobility of the cannula.

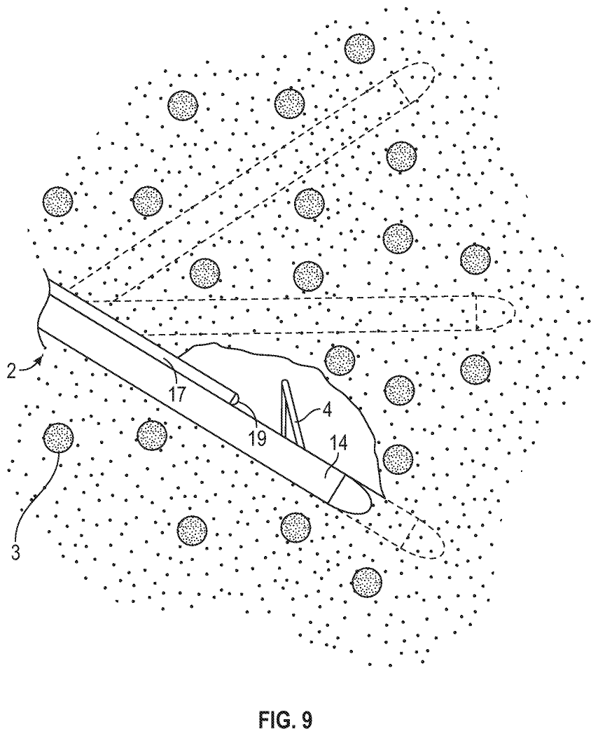

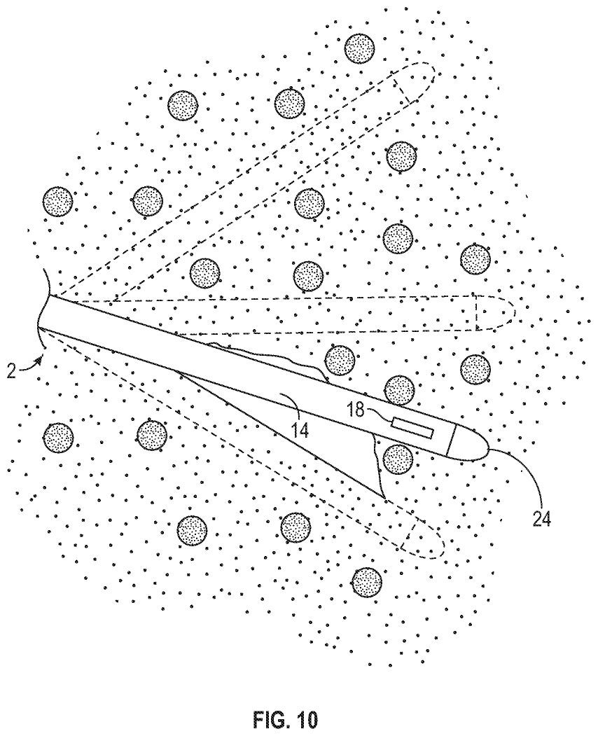

The cutting element 4 of the present invention permits the user to cut tissue adjacent to the liposuction wand. In this manner, the user can position the liposuction wand in a tunnel adjacent to fat deposits that the user desires to remove but cannot reach. The cutting element 4 is positioned adjacent to the area where the user desires to remove additional fat deposits as shown in FIG. 8. The cutting element 4 is then expanded and moved proximally to cut tissue and, in particular, the septae adjacent to the body as shown in FIG. 9. The cutting element 4 is then retracted to prevent undesirable tissue damage. The body 14 is then moved so that a distal tip 24 is positioned in the area where the tissue has been cut so that the user may access the tissue area adjacent the passageway using the same incision site, as shown in FIG. 10. Although not shown, the cutting element 4 can also be distally-oriented, as opposed to proximally-oriented as shown in FIGS. 8 through 10, in which case it is moved distally to cut tissue.

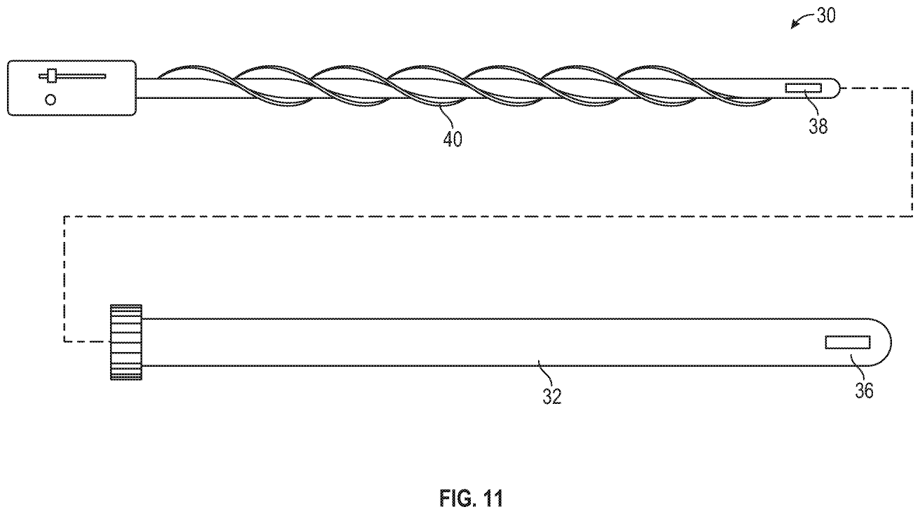

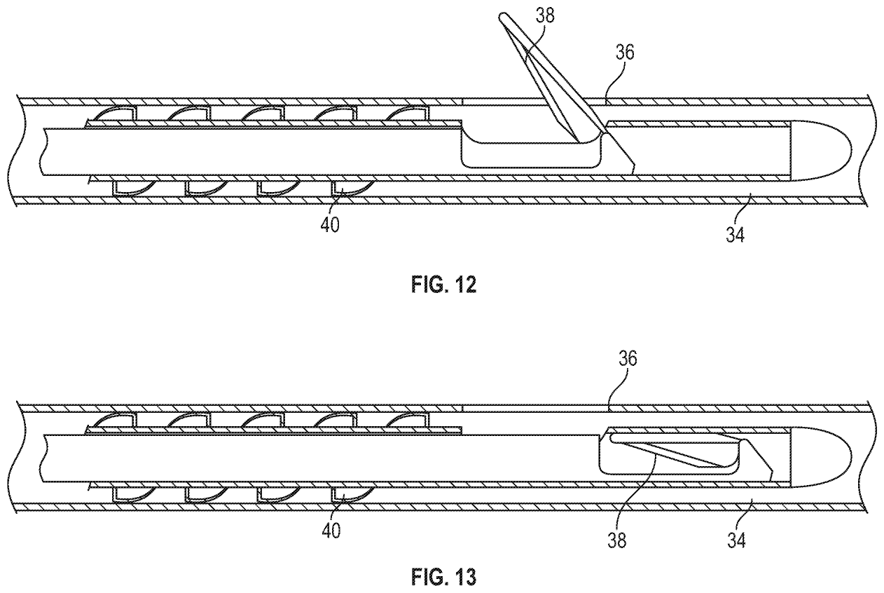

Referring to FIGS. 11-13, another device 30 is shown for cutting tissue which may be used in combination with a separate liposuction cannula. The device 30 includes a body 32 having a suction lumen 34 and one or more suction openings 36 which are used to remove fat and other tissue as described above. The device 30 also includes a cutting element 38 positioned inside the suction lumen 34. The cutting element 38 is movable from a position contained within suction lumen 34 to a position outside the suction lumen 34. The cutting element 38 may also be used to enhance withdrawal of the material through the suction lumen 34. To this end, the cutting element 38 may include a screw-like exterior surface 40 which enhances withdrawal of material when the exterior surface 40 is rotated. The cutting element 38 is similar to the cutting element 4 and the description of the cutting element 4 is applicable to the cutting element 38.

An advantage of the device 30 of FIGS. 11-13 is that the cutting element 38 may be advanced and withdrawn within the suction lumen 34 as needed. This permits the user to undertake a conventional liposuction procedure and, at the appropriate time, the user may advance the cutting element 38 within the suction lumen 34 so that that tissue may be cut. The cutting element 38 may also be advanced into the suction lumen 34 to enhance withdrawal of the tissue by rotating the exterior screw-like surface 40 to draw material proximally. The cutting element 38 may also be completely removed from the cannula 32 as well.

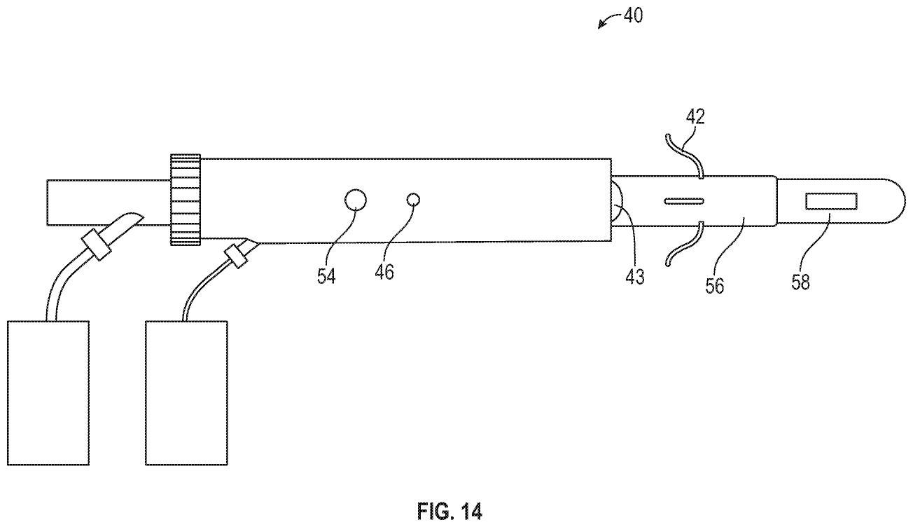

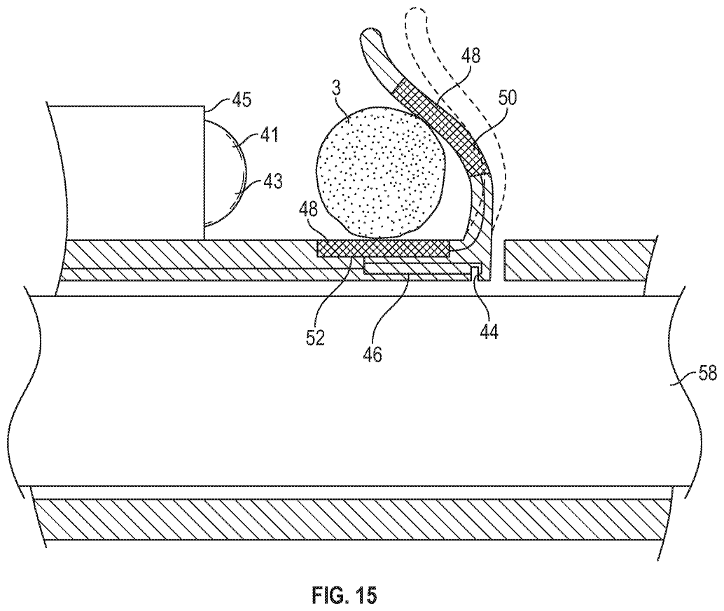

Referring to FIGS. 14-16, another tissue cutting device 40 is shown. The tissue cutting device 40 has one or more elements 42 which can be deflected as the cutting device 40 is moved through tissue. The embodiment of FIG. 14 has four elements 42 positioned at 90 degrees to one another around the body of the device 40. Spacing the elements 42 in this manner reduces the need to orient the device 40 in order to capture septae. When fewer elements 42 are used, the element(s) 42 are oriented to capture tissue which extends between the skin and muscle when moved through tissue. The element 42 loosely captures material with a hook-like structure. The element 42 can be deflected from the solid line position to the dotted line position of FIG. 15. Deflection of the element 42 may be used to distinguish relatively robust structures, such as septae, from softer tissues, such as blood vessels and nerves. Softer structures, such as blood vessels and nerves, may not be rigid enough to deflect the element and may be able to slip around the end of the relatively open hook-like element 42. The hook 42 may have smooth surfaces and transitions to minimize trauma to tissue and permit softer tissues to flow around the hook if the tissue is not robust enough to deflect the element. The element 42 may take any other suitable shape such as a V-shape.

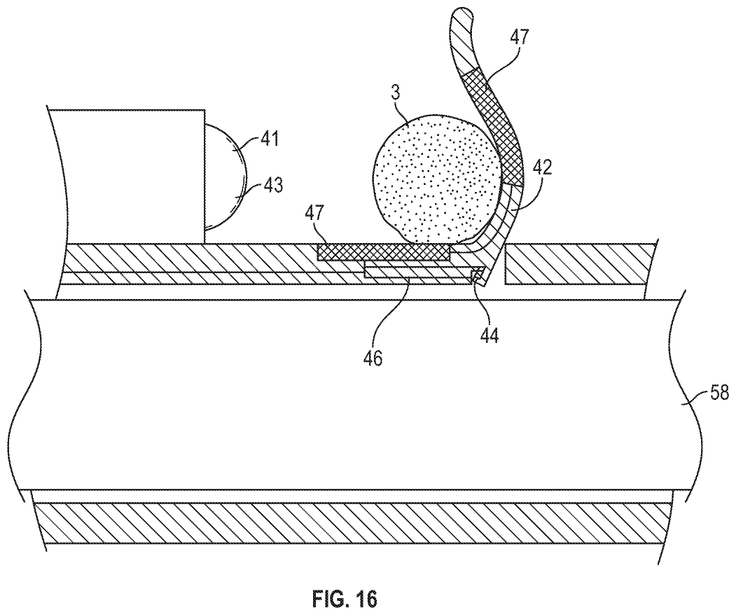

Deflection of the element 42 may be recognized in any suitable manner. For example, a first contact point 44 on the element 42 may move into engagement with a second contact 46 as shown in FIG. 16. Of course, any other method of determining whether the element 42 has been deflected may be used. Furthermore, it is understood that deflection of the element 42 represents a threshold force required to move the element 42. As such, any other force-sensing element could be used including a piezoelectric element or a spring. The device 40 may also include a visualization device 41, such as an endoscope 43, to observe the tissue which has been trapped by the element 42. In this manner, the user may inspect the tissue which has been trapped prior to cutting. If the user does not want to cut the tissue which has been trapped, the user may simply manipulate the device 40 by gently rotating and/or advancing the device 40 to release the tissue which has been trapped. The endoscope 43 may be integrally formed with the device 40 or contained within a lumen 45.

If the element 42 is deflected as shown in FIG. 16, an indicator 46 on the device 40 may indicate to the user that the element 42 has been deflected. At this time, the user may activate a cutting mechanism 46 to sever or cut the material captured by the element 42. The cutting mechanism 47 may be any suitable cutting mechanism with FIG. 15 showing an RF cutting element 48 but may also be an ultrasound element which delivers ultrasonic energy to cut the tissue. The RF cutting element 48 has a first electrode 50 and a second electrode 52 for bipolar RF but may be configured for monopolar RF as well. The device 40 may operate to automatically activate the cutting mechanism 47 once the element 42 has been deflected or may require the user to activate the cutting element 47 using a push-button 54 or other suitable actuator.

The element 42 may be coupled to a sleeve 56 which extends over a liposuction cannula 58. As such, the user may elect when, and if, the element 42 is used if at all. The sleeve 56 also permits the user to move the cutting element 48 longitudinally along the cannula 58 so that the cutting mechanism 48 may be used to selectively release septae without having to move the cannula as described above.

The element 42 may also be used for blunt dissection of tissue. The element 42 will capture and cut the septae by application of sufficient force to rupture the septae. The user may inspect the tissue using the visualization device 41 to distinguish septae from other tissue structures as discussed above before cutting tissue. Without the use of a visualization device, the user may also tug on the captured tissue to see if it is strongly connected to the skin. By evaluating the effect on the skin surface, tissues desired to be cut can be differentiated from those which are undesirable to cut.

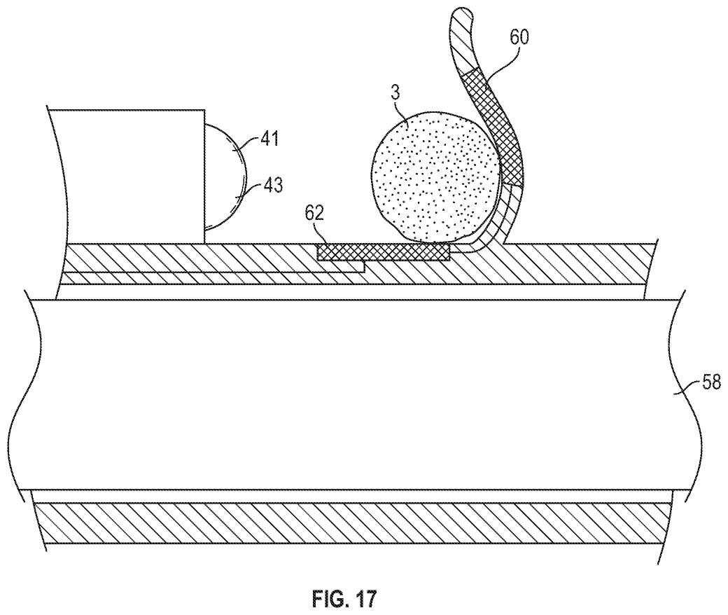

Another way to distinguish structures desired or permissible to be cut such as septae and fat from tissues undesirable to affect, such as blood vessels and nerves, is to differentiate structures by their electrical characteristics. Referring to FIG. 17, a first electrode 60 and a second electrode 62 can be used in a bipolar configuration to measure electrical impedance of captured tissue. The electrode or electrodes 60, 62 can also be configured for monopolar measurements, which would require a reference electrode elsewhere on the subject. The same electrodes 60, 62 can be used to enact the cutting of the tissue. Operation and use of the elements 60, 62 may be the same as described above in connection with FIG. 11. For example, the indicator 46 may be used to determine whether the electrical impedance measured by the electrodes 60, 62 is within a threshold range which may help to distinguish blood vessels and nerves from septae. The device may operate to automatically activate cutting of the tissue or may require the user to activate the cutting element as described above.

In some embodiments, the devices described above can be modified to cut septae without fat removal capabilities. In some embodiments, the devices comprising fat removal functionality can be used solely to cut septae within a treatment area.

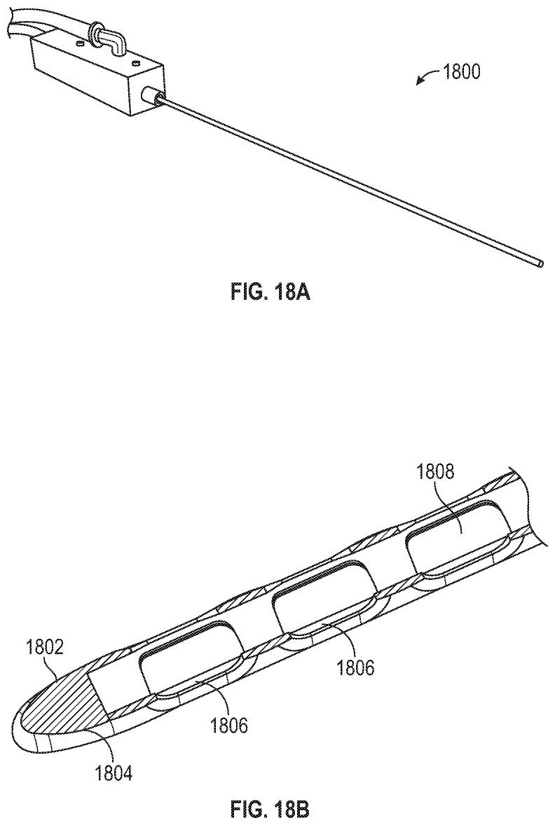

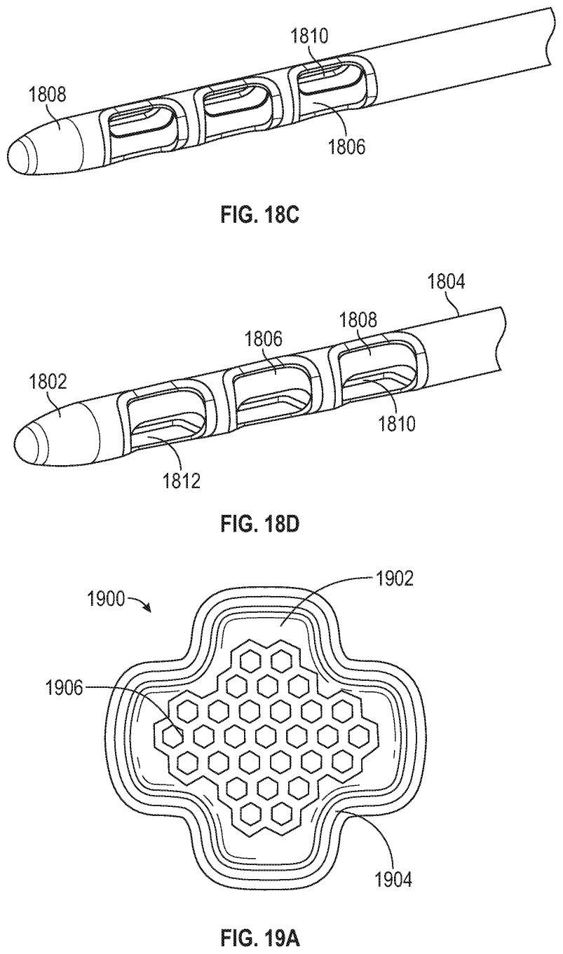

FIGS. 18A-18D depict another embodiment of a cutter 1800, shown in FIG. 18A. FIGS. 18B-D show a distal end of the cutter shaft 1802. The cutter shaft comprises an outer sheath 1804. The outer sheath 1804 comprises a generally cylindrical shape. The outer sheath 1804 comprises one or more apertures 1806 along a length of the sheath 1804. FIGS. 18B-D show three generally rectangular shaped apertures 1806 arranged in a direction parallel to the longitudinal axis of the sheath 1804. Other shapes (e.g., circular, ovular, etc.) and configurations (e.g., around the circumference of the shaft, linearly along the shaft and around the circumference of the shaft, etc.) are also possible.

The shaft 1802 also comprises an inner sheath 1808 comprising apertures 1810 arranged to line up with outer sheath apertures 1806. The edges of the inner/outer sheath apertures 1810 comprise a cutting surface. The inner sheath 1808 is configured to rotate relative to the outer sheath 1804. When the shaft 1802 is properly in place at a treatment site where cutting is desired, the inner apertures 1810 and outer apertures 1806 can be at least partially aligned. Vacuum can be applied to the lumen 1812 (best shown in FIG. 18D) of the cutter shaft 1802, drawing the tissue (e.g., selected septae) to be cut through the apertures 1806, 1810, and into the lumen 1812. Rotation of the inner sheath 1808 relative to the outer sheath 1804 causes any tissue previously drawn into the lumen 1812 to be cut.

The cutter 1800 can be used with the visualization devices (e.g., visualization device 41) or any of the septae recognition methods and devices described above.

In some embodiments, an inner sheath 1808 with apertures 1810 can be translated longitudinally, parallel with the axis of the cannula, instead of rotating, yet with similar results. Applied vacuum invaginates tissue through the apertures 1810 and 1806 when aligned. As apertures are closed, the tissue is cut and aspirated through the length of the catheter.

The cutting plane and position of the cutters may also be shown by using a light emitting element positioned at the distal end of the cutting tool at the cutting location. In some embodiments, the light emitter may be the end of a polished or cleaved fiber. Light may be directed in a direction orthogonal to the cutting plane toward the skin surface. The intensity and/or focus of the visible light would provide information regarding the angle of the cutting plane relative to the skin surface. The light source may be a red LED positioned in the handle and sending a visible signal through a fiber optic cable or light pipe, positioned in the wall of the tool and down its length.

The cutting plane can also be identified and oriented through features such as flats, ridges, and shape and/or other features of the cannula handle.

In some embodiments, the mechanical cutting action itself may be selective in nature. The sharpness of the blades or blade configuration (e.g. serrations) may be designed to ensure only lower elasticity elements may be cut. Septae in areas of cellulite will be under different levels of strain and therefore be closer or farther away from their elastic limits (taut). It is also possible that dimple causing septae are thicker elements as noted in MRI studies in the background section above. Both taut and thick septae will oppose the cutter opening force more than “loose” or “thin” septae. Therefore, a spring action used to expose the cutters may be dialed in such that enough apposition force is present to cut, or partially sever, taut or thick septae.

### Splint or Patch

A patch or splint can be used in combination with the cutting of the septae to control the healing of the tissue and prevent the treated dimples from reforming. While cutting the septae described herein has been in reference to subcutaneous cutting, the use of the splint in conjunction with other minimally invasive septae damaging/cutting/releasing technologies (subcutaneous or cutaneous), such as laser, RF, or HIFU is anticipated. The splint can ensure a homogenous topography of the dermal surface is maintained as the skin remodels smooth to a state of force equilibrium. Note that the terms patch and splint are used interchangeably through the disclosure.

The splint is flexible enough to conform to and move with the skin, and rigid enough to hold the local skin deformities, such as dimples, flat and in place, while healing into a remodeled smooth shape. Initial designs included a thin, flexible pad. Such a pad provides good contouring to skin and movability along with the skin through different body positions; however, the pad may not provide sufficient rigidity to hold skin in targeted treatment area to the desired shapes. Dimples may reappear with a thin, very flexible patch. Thicker patches were also tested. It was found that thicker patches that were overly rigid did not move with the skin well, causing high stress concentrations at the edges of the patch. The high stress concentrations can lead to dehiscing of the tissue under those areas. Thus, it was discovered that a proper balance and transitioning between flexible and rigid areas can lead to a desired result of the treatment.

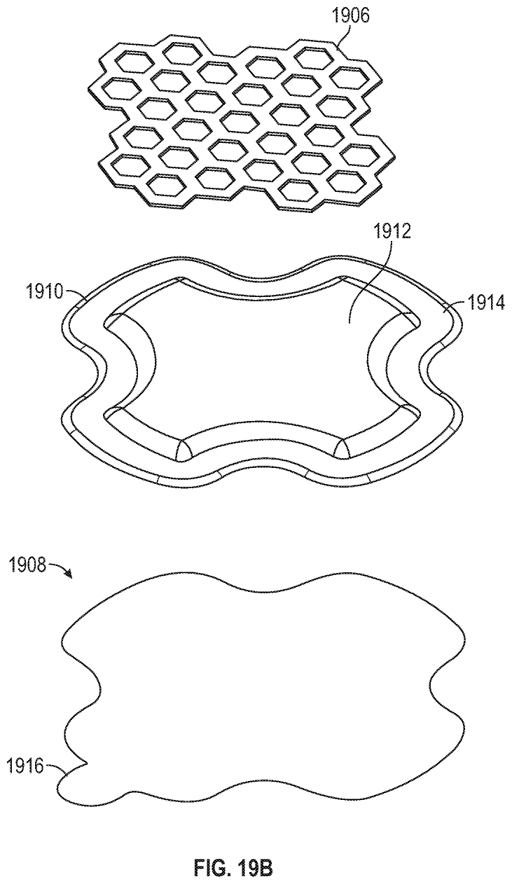

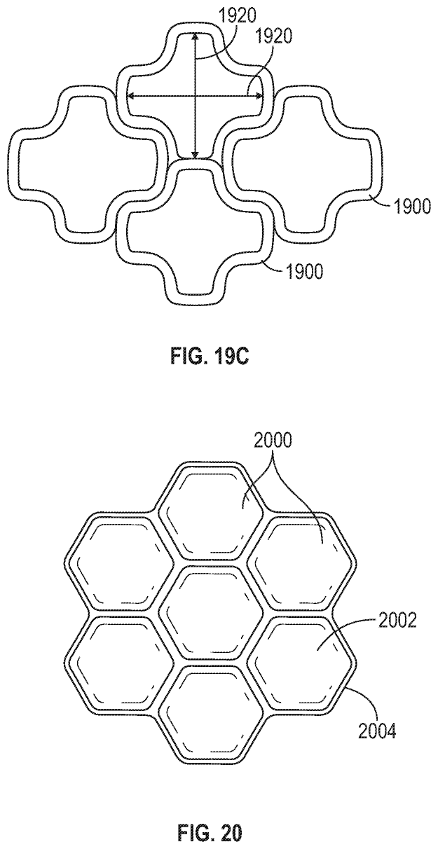

FIG. 19A shows an embodiment of a patch 1900. It was found that a pad in combination with an inner backbone provides the desired combination of flexibility and rigidity. The patch comprises a thicker central portion 1902 and thinner edges or border 1904. At least a portion of the central portion 1902 comprises a backbone 1906.

These features are shown in greater detail in the exploded view of FIG. 19B, which shows a backing 1908, a pad 1910, and the backbone 1906. Together, these components form the patch 1900. The backbone 1906 is shown as comprising a honeycomb structure, but other configurations (e.g., circular openings, diamond shaped openings, square shaped openings, etc.) are also possible. The rigidity of the patch can be adjusted by adjusting the size and spacing of the openings in the backbone, the material, and/or the thickness. In some embodiments, the backbone is solid. In some embodiments, the openings can be about 0.01 in. (or about 0.005-0.015 in. 0.0075-0.0125 in. in diameter, etc.). In some embodiments, a thickness of the backbone can be about 0.02 inches (or about 0.01-0.03 inches, about 0.15-0.25 in). The backbone is positioned within the pad 1910. In some embodiments, an area of the backbone can comprise about 10% - 90% (or about 10-20% or about 20-30% or about 30-50% or about 50-70% or about 50-80% or about 60-80% or about 70-80% or about 80-90%, etc.) of the area of the pad. In some embodiments, the backbone comprises polycarbonate. Other materials are also possible (e.g., fiber, other polymer, etc.).

As noted above, the backbone is positioned within the pad 1910. The pad comprises a flexible material, such as an elastomer. In some embodiments, the pad comprises a silicone elastomer. Other materials are also possible (e.g., polyurethane, polyisoprene, neoprene, rubber, etc.). In some embodiments, the patch comprises a clear material, which can allow for proper alignment/targeting of the treatment area to be confirmed as well as adherence to the skin to be observed through the patch. The material can have a durometer of about 50A (or about 30-60A, or about 45-60A, etc.). The material can have a tensile strength of about 5.3 MPa (or about 4.7-5.8 MPa or about 5.1-5.5 Mpa, etc.). The pad has a thicker central portion 1912 and a thinner edge portion 1914. The difference in thickness can help reduce the stress concentration at the edges of the patch, reducing the likelihood that the edges of the patch will cause dehiscing of tissue that the edges will detach from the tissue. The central portion can have a thickness of about 0.13 in (or about 0.08-0.18 in, or about 0.11-0.15 in, etc.). The border can have a thickness of about 0.031 in (or about 0.021-0.041 in, or about 0.026-0.036 in., etc.). The border of the central portion and the border of the patch can have rounded or smooth edges to provide a more comfortable user wearing experience. The backbone can be embedded within the pad so that it is parallel to the surfaces of the pad and is positioned centrally within the thickness of the pad. In some embodiments, the backbone can be offset with respect to the center of the thickness of the pad. Adjusting the thickness of central area and/or the edges of the pad can adjust the rigidity/flexibility of the patch and the transition to the skin.

In some embodiments, a thicker patch, with or without a separate back bone, may be patterned with directional cuts or channels to preferentially direct more/less patch flexibility. In some embodiments, a patch, with or without a separate back bone, may have additional material added to the patch to preferentially direct more/less patch flexibility. For example, softer or stiffer strips of material (either the same or different from the patch) can be attached to the patch to make it more flexible in one direction than another. A single embodiment can include any combination of features described herein as affecting flexibility of the patch.

In some embodiments, the splint can be filled with a UV curable gel that can be selectively hardened to maintain the splint's shape and/or enhance the stiffness or flexibility of the spling.

Finally, the patch comprises a backing 1908. The backing is an adhesive layer applied to the patch and covered with a layer of release paper, that when removed, exposes the adhesive in preparation for application to the skin. The backing can comprise an area and shape generally equivalent to the area and shape of the pad. The backing can include a tab 1916 that aids in removal of the release paper. In some embodiments, the backing comprises PTFE. Other materials (e.g., HDPE, poly ethylene, BO PEP, BOP, Kraft paper, or other low surface energy material) are also possible. The backing can comprise a thickness of about 0.0010 in (or about 0.003-0.03 or about 0.004-0.0020 in., etc.). The backing comprises an adhesive, for example, a pressure sensitive adhesive (PSA) configured to adhere the patch to a user's skin. The adhesive can be a medical grade adhesive. The backing can advantageously provide a smooth surface for the adhesive, creating a better bond line between the patch and the skin. The adhesive (e.g., PSA) can have a peel adhesion of about 500 g/cm, about 700 g/cm, about 500-700 g/cm, about 400-800 g/cm, etc. The adhesive (e.g., PSA) can have a shear of about 15 kg/6.25 cm², about 16 kg/6.25 cm², about 15-16 kg/6.25 cm², about 14-17 kg/6.25 cm², about 13-18, kg/6.25 cm², etc. The adhesive can have a thickness of about 0.005 in. (or about 0.002-0.007, or about 0.004-0.006, etc.).

In some embodiments, a UV curable gel can be used to secure the patch to the tissue. The gel can be placed between the skin and the patch, and cured to secure it in place.

The patch can comprise the shape shown in FIGS. 19A-19C, resembling a rounded plus sign. The shape comprises a circle with four rounded cuts out spaced approximately 90° around the circle. Other shapes are also possible.

The patch 1900 can be used individually or can be arranged in a matrix to cover a larger area, as shown in FIG. 19C. The rounded plus shape of the patch allows for ease in nesting multiple patches together to create a grid of patches. Other patch shapes allowing this sort of placement are also possible. A clinician can orient the patch or series of patches to best address the tissue defects at the treatment area. The rounded plus shape, in particular, allows for a greater length along the axes 1920 shown in FIG. 19C, allowing for the treatment of a larger length area, while also allowing for ease in nesting with other patches to form a grid pattern. Arranging a series of smaller patches in a grid pattern allows for greater adherence to the skin that a similarly sized larger patch. A larger patch may require increased flexibility to conform to a larger area of skin, which may decrease the efficacy of the patch, as it may not be rigid enough to properly hold the desired shape of the skin.

The size of the patch can be selected based on the desired treatment area. In some embodiments, the patch (e.g., the patch shown in FIGS. 19A-19C) has a radius of about 2 in. (or about 1-3 in., or about 1.5-2.5 in., etc.).

FIG. 20 shows a matrix of smaller patches 2000 that can be used to achieve more focused remodeling of the skin. A smaller patch does not need to be as flexible as a larger patch as it conforms to smaller area of skin than a larger patch. As such, a smaller patch can be designed without the inner backbone. In some embodiments, the smaller patch does comprise the inner backbone. For smaller patches, additional “transition” patches of varying thicknesses may be applied adjacently to reduce the skin tension of a smaller, stiffer patch. Smaller patches can include shapes roughly ½ the surface area of the larger patches. In some embodiments, each patch 2000 can be separate, allowing the patches to be arranged as indicated by the skin to be treated. Each patch 2000 comprises a thicker central portion 2002 and a thinner edge portion 2004. The thickness of the central portion 2002 and edge portion 2004 can be similar to that described above with respect to the larger patch of FIGS. 19A-C.

In some embodiments, the patch comprises holes extending through the thickness of the patch. The holes can allow for sweat, water, water vapor, etc. from the tissue to escape, improving the durability and efficacy of the adhesive. The holes can also allow for external vacuum to seat the skin firmly against the splinting surface.

In another smaller patch embodiment, additional material, the same or different than the smaller patch material, can be applied and adhered to all or part of the matrix, to provide additional stiffness/reduced flexibility in a desired direction.

Finally, a slippery cover may be applied over single or multiple patches to allow clothing to slip over the treatment area and not impart an external load to the treatment site.

### Applicator

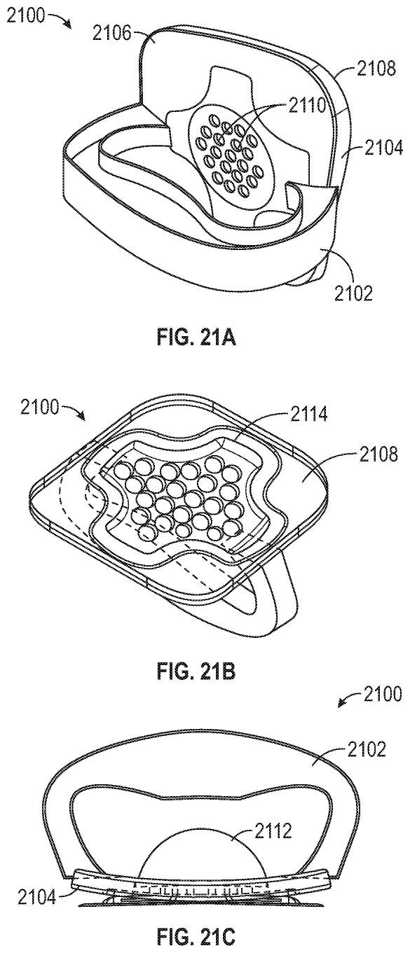

In some embodiments, the patch can be applied by the clinician directly by hand. In some embodiments, an applicator is used to apply the patch. FIGS. 21A-C show various views of an embodiment of an applicator 2100. As shown in the perspective view of FIG. 21A, the applicator 2100 comprises a handle 2102 and a body 2104. The body comprises a top surface 2106 and an applicator surface 2108. The applicator surface can comprise indentations 2114 shaped to mate to the patch (e.g., the top of the patch). Apertures 2110 can extend from the top surface 2106 through to the applicator surface. The apertures 2110 can be configured to transmit applied vacuum from the top surface to the applicator surface to hold a patch in place during application. The top surface 2106 can accommodate features for mounting a vacuum source such as a vacuum bulb 2112 (shown in FIG. 21C) or other feature configured to deliver vacuum to the apertures 2110. The handle 2102 can extend across a length of the applicator, allowing a clinician to grip the handle 2102 and firmly apply force to the applicator. In some embodiments, the body 2104 is curved along its length, as shown best in FIG. 21C. This curvature allows a clinician to press down and roll the applicator along the skin during application. The curved surface helps concentrate the applied force to the portion of the applicator surface/patch contacting the skin. This increased force may be more effective in securing the patch to the skin. The increased force may also be more effective in activating PSA in patch embodiments comprising PSA as an adhesive. The radius of curvature can be about 10-14 in.

In some embodiments, the applicator comprises an optically transparent material to allow a clinician to ensure a secure adherence between the applicator and the patch.

In some embodiments, the applicator comprises one or more features that aid in creating a matrix of patches. For example, the applicator surface may comprise additional indentations shaped to mate with adjacent, previously placed patches, allowing a clinician to determine proper placement of a matrixed patch. The applicator can also comprise alignment marks to help align an attached patch to previously placed patches.

Pressure sensitive or temperature sensitive films or temperature/force sensors may be present on the applicator, patch, or targeting device to show the significant peaks and valleys present in the cellulite. Peaks would show up as a high force or a high temperature region when moderate downward pressure is applied with the positioning tool. These sorts of features can help prevent excessive force when applying the patch.

### Method of Reducing the Appearance of Cellulite

A clinician can determine a desired treatment area. This determination can be made by the naked eye upon a visual inspection of the skin. This determination can also be made using the targeting devices described with respect to FIGS. 2A-2C. The viewing portion can be pressed against a potential treatment area, exaggerating and/or highlighting any existing dimpling. The viewing can be done with the patient standing, sitting or laying down to assess if there is any directionality to the skin's substructure that should be addressed with subcision. Once a treatment area has been selected, the clinician may use a cutter, such as those described with respect to FIGS. 3-18B, to selectively cut the septae at the treatment area. In some embodiments, the targeting device is again used to press down on the treatment area during the treatment and assess whether sufficient reduction in dimpling has occurred. Once the clinician is satisfied with the septae cutting, a patch is applied over the treatment area. The patch can be adhered to the applicator using vacuum. This may securely fit the patch within the indentations on the applicator surface. The applicator may then be pressed or rolled onto the skin in the treatment area. Any applied vacuum can be discontinued during the rolling or pressing process. External vacuum maybe applied to an applied patch if necessary to draw the skin tight to the patch. Patches comprising an optically clear material allow for assessment of proper treatment and/or targeting.

In some embodiments, most or all of the septae at a treatment area are treated (e.g., cut, damaged, released, etc.). In some embodiments, selective treatment within a treatment area results in cutting, releasing, damaging, or otherwise treating only one or more selected septae within a treatment area, leaving one or more unselected septae within the treatment area untreated. It has been demonstrated that cutting every septae in a treatment area has some effect, but not necessarily the desired effect caused by selective subcision. The improved results are, at least in part, effected by the more localized inflammation caused by selective treatment versus the more widespread inflammation caused by treatment of all septae in a treatment area. The splint can prove more effective in remodeling skin exhibiting the more localized inflammation.

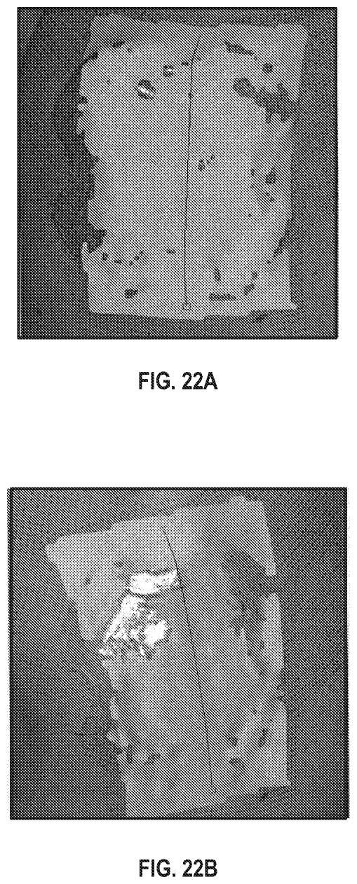

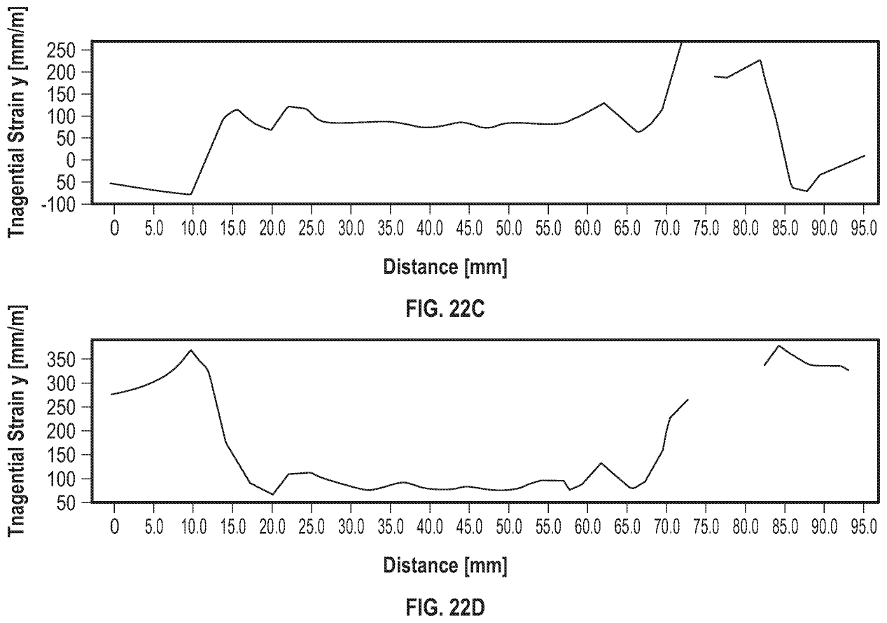

FIGS. 22A-22D show temperature plots for an applied patch in a compressed position (FIGS. 22A and 22C) and an extended position (FIGS. 22B and 22D). These plots shows how the mid-section of the patch, under both compression and extension, maintains stability while the thin border or edge serves as strain relief to the skin surface.

In some embodiments, the patch is worn for about 4-6 weeks following treatment. In some embodiments, the patch may be worn for more or less time. The patch may fall off and need to be replaced during this period.

In some embodiments, the patch is worn under compression garments that may aid in reducing inflammation caused by the treatment. In some embodiments, the patch may be incorporated into a compression garment. The patch may be attached to the inside of the compression garment. In some embodiments, the patch does not comprise adhesive and is instead held in place over the skin by the compression garment.

In some embodiments, with the applicator attached to the top surface of the patch and the patch adhered to the skin, the assembly may be used to pull upward applying tension to skin. This tension is counteracted by the septae connecting the dermis and fascia. This stress may be applied in such a way as to assist in cutting. Septae under greater stress/strain may be more easily cut. The upward force applied using the splint and positioning tool may provide for tighter target septae allowing for easier cutting.

The devices and methods disclosed herein can be used in combination with other technologies and treatments such as topical agents, manual or automated message, targeted liposuction, targeted ultrasound, sub dermal infections, targeted RF, selective subcision and Laser. Many of these have seen success in select patient populations and cellulite topography.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A patch for treatment of cellulite, comprising
a flexible pad comprising a central portion and a border portion, the border portion thinner than the central portion;
a backbone embedded within the flexible pad, the backbone more rigid than the flexible pad; and
an adhesive configured to adhere the patch to skin,
wherein the patch is configured to be adhered to the skin, thereby holding the skin in a remodeled configuration during healing.

2. The patch of claim 1, wherein the backbone comprises a sheet of material comprising a plurality of apertures.

3. The patch of any of claims 1-2, wherein the backbone comprises a honeycomb structure.

4. The patch of any of claims 1-3, wherein the flexible pad comprises a silicone elastomer.

5. The patch of any of claims 1-4, wherein the backbone comprises a polycarbonate.

6. The patch of any of claims 1-5, wherein the adhesive comprises a pressure sensitive adhesive.

7. The patch of any of claims 1-6, wherein the patch is shaped to be positioned adjacent to other same shaped patches to form a matrix.

8. The patch of any of claims 1-7, further comprising a backing positioned between the pad and the adhesive.

9. The patch of claim 8, wherein the backing comprises PTFE.

10. The patch of any of claims 1-9, further comprising a plurality of apertures extending through the patch.

11. The patch of any of claims 1-10, wherein a thickness of the flexible pad is about 0.11-0.15 in.

12. The patch of any of claims 1-11, wherein a thickness of the border is about 0.026-0.026 in.

13. A method of reducing an appearance of cellulite comprising
adhering a patch to skin over a treatment area of a user, the treatment area comprising released septae; and
maintaining a configuration of the skin with the patch.

14. The method of claim 13, wherein the released septae are subcisioned or released through cutting or through electromechanical means.

15. The method of claim 13, wherein the patch comprises a flexible pad comprising a central portion and a border portion, the border portion thinner than the central portion and a backbone embedded within the flexible pad, the backbone more rigid than the flexible pad.

16. An applicator for applying a patch to skin, the applicator comprising
a handle; and
a body attached to the handle, the body comprising a top surface and a bottom applicator surface, the body comprising a plurality of apertures extending from the top surface to the bottom applicator surface, the bottom applicator surface curving towards the handle.

17. The applicator of claim 16, further comprising one or more indentations on the applicator surface shaped to receive the patch.

18. The applicator of any of claims 16-17, further comprising a feature on the top surface configured to receive a vacuum source.

19. The applicator of any of claims 16-18, wherein the body comprises an optically clear material.

20. The applicator of any of claims 16-19, wherein the applicator is configured to apply the patch of any of claims 1-12.

21. A method of applying a patch to skin, comprising
providing an applicator comprising a body and a handle extending across the body;
attaching a patch to a curved bottom applicator surface of the body;
gripping the handle;
applying downward force to the applicator; and
rolling the applicator across the skin.

22. The method of claim 21, wherein attaching the patch to the curved bottom applicator surface of the body comprises applying vacuum to the patch.

23. The method of any of claims 21-22, wherein attaching the patch to the curved bottom applicator surface of the body comprises placing the patch within indentations of the bottom applicator surface.

24. The method of claim 23, wherein the indentations are shaped to match a shape of the patch.

25. A targeting device for determining a cellulite treatment location, comprising
a handle; and
a viewing portion attached to the handle, the viewing portion comprising a sheet of optically clear material.

26. The targeting device of claim 25, wherein the handle comprises indentations shaped to receive a user's fingers.

27. The targeting device of any of claims 25 and 26 where the viewing portion has holes through which a therapist can use a marking pen to mark the treatment location.

28. The targeting device of any of claims 25-27, wherein the sheet is flat.

29. The targeting device of any of claims 25-27, wherein the sheet is curved.

30. A method of determining a skin treatment location, comprising
holding a handle of a targeting device; and
pressing a viewing portion of the targeting device against a potential treatment site, the viewing portion comprising a sheet of optically clear material.

31. The method of claim 30, further comprising selecting the potential treatment site and treating a skin defect at the treatment site.

32. A method of reducing an appearance of cellulite in a subject, comprising
selecting a treatment site in the user;
cutting targeted septae at the treatment site; and
adhering a patch to the skin over the treatment site, the patch comprising a flexible pad comprising a central portion and a border portion, the border portion thinner than the central portion and a backbone embedded within the flexible pad, the backbone more rigid than the flexible pad.

33. The method of claim 32, wherein selecting a treatment site in the user comprises
holding a handle of a targeting device; and
pressing a viewing portion of the targeting device against a potential treatment site, the viewing portion comprising a sheet of optically clear material.

34. The method of any of claims 32-33, wherein adhering the patch to the skin over the treatment site comprises
providing an applicator comprising a body and a handle extending across the body;
attaching the patch to a curved bottom applicator surface of the body;
gripping the handle;
applying downward force to the applicator; and
rolling the applicator across the skin.

35. The method of claim 34, wherein attaching the patch to the curved bottom applicator surface comprises applying vacuum to the patch.

36. A method of claim 35, wherein an external vacuum can be used to pull the tissue into the patch through holes in the patch.

37. The method of any of claims 32-35, further comprising adhering a second patch adjacent to the patch, the second patch placed such that edges of the patch and second patch align with one another.
